(19) United States
(12) Patent Application Publication
(10) Pub. No.: US 2020/0078485 A1
(43) Pub. Date: Mar. 12, 2020

(54) PROVIDING USER-SELECTED FRAGRANCE BLENDS

(71) Applicant: NOUSTIQUE PERFUMES, S.L., Barcelona (ES)

(72) Inventors: Alvaro SUAREZ IRIBARNE, München (DE); Jakub Jan JEDLINSKI, Neubiberg (DE); Hugo LASALA ALONSO, Zaragoza (ES); Manuel GIMENO ASIN, Zaragoza (ES); Javier SANZ NAVAL, Zaragoza (ES); Aida RODRIGUEZ GARCIA, München (DE)

(21) Appl. No.: 16/469,576

(22) PCT Filed: Dec. 1, 2017
(86) PCT No.: PCT/EP2017/081251

§ 371 Date: Jun. 13, 2019

(30) Foreign Application Priority Data
Dec. 16, 2016 (EP) 16382610.0
Dec. 16, 2016 (ES) P201631602

(51) Int. Cl. A61L 9/03; A61L 9/12; A61L 9/01

(52) U.S. Cl. CPC A61L 9/035; A61L 2209/133; A61L 9/01; A61L 9/125

(57) ABSTRACT

A method according to the invention for providing a user-selected fragrance blend (FB) by a fragrance dispensing apparatus (1) comprises querying to select, via a user interface (14), a fragrance reservoir (CS1-CS3) containing several aroma-compounds (F1-F5) belonging to a common scent family, and querying to select, via the user interface (14), at least one other fragrance reservoir (CS1-CS3) containing at least one aroma-compound (F1-F5) belonging to another scent family, until aroma-compounds (F1-F5) belonging to all levels of a scent pyramid have been selected, extracting the aroma-compounds (F1-F5) from the selected fragrance reservoirs (CS1-CS3), and dispensing the extracted aroma-compounds (F1-F5) as a fragrance blend (FB). A fragrance dispensing apparatus (1) according to the invention is adapted to perform the method according to the invention. The invention is particularly advantageous for operation by an end user of the fragrance blend.

The invention relates to a method for providing a user-selected fragrance blend by a fragrance dispensing apparatus, the method comprising extracting aroma-compounds from selected fragrance reservoirs and dispensing the extracted aroma-compounds as a fragrance blend. The invention also relates to a fragrance dispensing apparatus for dispensing a user-selected fragrance blend, comprising several fragrance reservoirs containing aroma-compounds, a user interface for selecting at least two fragrance reservoirs containing aroma-compounds to be blended into the fragrance blend, at least one extraction means for extracting the aroma-compounds from the selected fragrance reservoirs, and a fragrance dispenser for dispensing the fragrance blend comprising the extracted aroma-compounds. The invention is particularly advantageous for operation by an end user of the fragrance blend.

JP 2005 194419 A discloses a perfume capsule being stable to sodium hydrogen carbonate, sodium sulfate, or the like, not causing deterioration thereby and free from deterioration and capable of retaining good aroma over a long time when charged into water kept at a relatively high warm temperature of 38 to 42° C. JP 2005194419 A further discloses a bathing agent in which the perfume capsule is formulated. The perfume capsule is obtained by encapsulating a perfume ingredient by using a modified pregelatinized starch as an encapsulating material. The modified pregelatinized starch comprises, as at least a part, at least one kind of starch of modified pregelatinized starches forming an aqueous solution having >=50 cps viscosity when measuring the viscosity at 30° C. after heating the starch for 5 min at 80° C. in a form of 20% aqueous solution. The bathing agent is obtained by formulating the perfume capsule.

CN 2 303 507 Y relates to a perfume capsule which comprises an air capsule, multiple air microcapsules inside the air capsule, and perfume microcapsules inside the air capsule. The volume of the perfume microcapsule is smaller than the air microcapsule. Via squeezing the air, the air capsule can be broken, and the air microcapsule is simultaneously burst. During the burst of the air microcapsule, the perfume of the perfume microcapsule generates the atomizing effect. The perfume capsule has the advantages of good sealing, difficult volatilization during storage, convenient use, and portability.

DE 42 39 082 C1 discloses a blister-pack accommodating very aromatic perfume. On one side of the surface piece at least one small closed container is arranged containing a portion of perfume and made of a material which can be manually destroyed. The container is a one-piece bowl-shaped raised formation of plastics foil, the underside of which is closed by a closure piece forming or supporting the adhesive layer. The closure piece is formed by a foil extending over the surface of the container and made of plastic. The adhesive layer is covered by a withdrawal foil. Several separated perfume containers are provided distributed over the surface, each containing different perfumes.

WO 2010/021469 A2 relates to a portable perfume capsule which is used by breakage thereof. WO 2010/021469 A2 provides a portable perfume capsule which is easily carried and conveniently used anytime and anywhere. That is, a perfume bag is formed by heat-sealing two thin vinyl sheets so that the portable perfume capsule is broken with only power of fingers. Perfume is injected into the perfume bag and is scattered when the perfume bag is broken. The portable perfume capsule is easily carried and provides amusement in the breakage of the perfume capsule. In addition, the fragrance is provided and stress can be relieved during breakage of the perfume capsule.

NZ 607 641 A discloses a container system for liquids such as spray fragrances. The system includes a parent container and a child container. The parent container provides a first cavity for confining a liquid, and couples detachably to the child container for refilling the child container through a supply opening in the parent container. The child container can be used for instance as a travel pack in a handbag or hand luggage. Here the child container is a compact dispenser, comprising a bowl-shaped rigid container having a refill opening, which is kept closed by a valve unless the dispenser is connected to the parent container, and a dispensing opening. These openings are located apart from each other. A pump dispenses liquid from the dispense opening. In a particularly simple construction, the opening of the bowl is covered by a deformable membrane to form a closed dispense cavity, the cavity becoming mainly or completely evacuated as the dispensing means is operated. The membrane then relaxes again, filling the dispenser, when the dispenser is re-applied to the parent container.

CN 202 687 926 U discloses a perfume filling machine, which comprises a support, a towing bracket arranged in the support, a filling frame arranged at the top of the support, more than one injection device arranged on the filling frame, and a perfume box arranged on one side of the support, wherein a pump body is arranged on the right side in the filling frame, the pump body is connected with the perfume box through a conduit, and the pump body is connected with more than one water pipe. According to the utility model, multiple perfumes can be filled simultaneously, so that the filling efficiency is improved.

CN 202 400 818 U discloses a valve provided with a pipeline and applicable for fragrance sliding way filling. The valve comprises a valve, wherein a handle switch is arranged on the valve; the valve also comprises a pipeline, wherein the valve is arranged on the pipeline; the valve is closed when the handle switch is parallel to the pipeline; and the valve is opened when the handle switch and the pipeline incline. The valve provided with the pipeline is simple in structure and convenient to operate; the accident of fragrance leakage resulted from misopening caused by the collision of the handle when sliding downwards along the sliding way; the environment pollution and the cost are reduced; and meanwhile, the pipeline and the valve are fixedly connected, so that the installation time is reduced, the production efficiency is improved greatly, and the production cost is saved.

CN 201 099 609 Y discloses a filling machine for filling drink and flavour into liquid container (as glass bottle), which comprises a frame, a transmission system and a rotary platform. The utility model is characterized in that each filling valve is provided with a temporary liquid storage barrel, a rotary portion control system uses a wireless communication system (industrial wireless Ethernet) which comprises a positioning device, a probe, a wireless access end and a wireless client end, whose signal processing is completed by periphery control system and operation is processed on a touch screen. The filling machine has reasonable structure and high automatic degrees, without explosion and leakage, while the filling capacity has large adjusting range (50-2000 ml) and the filling accuracy is high as +/−0.5 ml in 500 ml.

U.S. Pat. No. 5,713,403 relates to a method which simplifies the preparation of packaging units which contain different filling products. It is characterized by the fact that to fill the containers according to a specified or selected filling formation with different filling products, i.e. optionally with at least a first or a second filling product, the filling machine is controlled by a control device so that, as a function of the filling formation, the filling positions dispense different products into containers which are unique and appropriate to each product.

U.S. Pat. No. 6,371,451 discloses a scent diffusion apparatus and method that contains individual original scents for a plurality of scents to be diffused, constructs information on position information of the contained original scents and information on various scents to be diffused in a database, and mixes the original scents of the desired scent properly at a desired point in time based on the scent information, to thereby spray the desired scent. The scent diffusion method includes providing a plurality of scent spraying units containing original scents, heating the original scents via a heater, evaporating the original scents, and diffusing the evaporated scent with the air via an air supply pump. Thus, a desired scent can be transferred to a selected subject under correct concentration. The scent diffusion apparatus uses scent cartridges containing the original scents, such as cartridges used in an existing ink-jet printer, and uses the same control commands as those of a general personal computer or ink-jet printer compatibly. Thus, the scent diffusion apparatus can control an amount of diffusion precisely, be manufactured in a compact fashion, and used in connection with a general purpose computer in which almost all kinds of operating systems supporting an ink-jet printer are incorporated.

US 2012/247613 A1 discloses a vending machine for dispensing a selected blend of perfumes in response to the deposit of a pre-selected sum of money includes a plurality of containers and a plurality of distinctive scents with one of the scents disposed in each of the containers. A mechanism for selecting one or more of the scents and an amount of one or more of the scents is provided. Further, the vending machine includes a mechanism for blending the selected scents in the selected percentages and providing a sample to a perspective customer. Further, the mechanism includes a slot receiving payment for a quantity of the selected blend together with a delivery process for providing a bottle of the selected perfume to the customer.

US 2016/045838 A1 discloses an aroma substance delivery device adapted to deliver an aroma substance exclusively to the nasal cavities of an individual, comprising the following elements: (i) a source of carrier gas flow; (ii) regulating means which receives the carrier gas flow and regulates its passage through a plurality of channels; (iii) downstream of the regulating means a plurality of aroma substance-containing cartridges, one being associated with each channel, and (iv) disseminating means located in close proximity to the cartridges and adapted to deliver the individual aroma substances from the cartridges to nasal cavities by means of a conduit; there being positioned in each channel on the upstream side and in close proximity to the origin of the channel a flow restrictor, the flow restrictor being configured such that the pressure drop across the flow restrictor is higher than the pressure drop across the rest of the device. The device is particularly useful as an element in conjunction with an audio-visual display, to provide a complete sensory experience.

It is the object of the present invention to at least partially overcome the problems associated with the prior art. It is a particular object of the present invention to provide a compact and cost-effective means for dispensing a fragrance blend in a highly flexible and user-friendly manner.

The object is achieved according to the features of the independent claims. Advantageous embodiments can be found, e.g., in the dependent claims, in the subsequent description and in the attached drawing.

The object is achieved by a method for providing a user-selected fragrance blend by means of a fragrance dispensing apparatus. The method comprises: querying to select, via a user interface, a reservoir (“fragrance reservoir”) containing several aroma-compounds belonging to a common scent family, and querying to select, via the user interface, at least one other fragrance reservoir containing at least one aroma-compound belonging to another scent family, until aroma-compounds belonging to all levels of a scent pyramid have been selected, extracting the aroma-compounds from the selected fragrance reservoirs, and dispensing the extracted aroma-compounds as a fragrance blend.

This method gives the advantage that it allows to blend a satisfactory fragrance blend with a reduced number of fragrance reservoirs. By ensuring that the fragrance blend comprises aroma-compounds from all levels of the scent pyramid, the fragrance blend has a well-rounded smell. Choosing the ingredients of the fragrance blend by scent family gives the further advantage that the relative olfactory strength of the scent families can be individually chosen. This, in turn, allows accurately adjusting the olfactory character of the fragrance blend by a user. As another advantage, this fragrance dispensing apparatus enables instantly creating custom-made or tailor-made fragrance blends. There is a high degree of flexibility in creating the fragrance blend since this is not restricted to choosing from ready-mixed fragrance blends. There is also a high degree of user-friendliness since this creating of custom-made or tailor-made fragrance blends can be performed by the user itself or in the presence of the user.

The fragrance blend is thus a blend of aroma-compounds from at least two fragrance reservoirs. Instead of using fragrance reservoirs containing only single aroma-compounds or using fragrance reservoirs containing different, already final fragrance blends, this invention realizes the idea to use fragrance reservoirs containing mixtures of multiple aroma-compounds (“aroma-compound cluster”) to be mixed or blended with at least one more aroma-compound—preferably with at least one more aroma-compound cluster—to produce a final fragrance blend.

The querying to select a fragrance reservoir comprises that the user interface waits for a user to select a certain fragrance reservoir. The method proceeds after the user has selected a fragrance reservoir.

For example, the user interface may comprise keys for inputting commands and/or selections and a display. The user interface may comprise a touchscreen.

An aroma-compound may be an essential oil. The aroma-compound may be one from the group of esters, linear-terpenes, cyclic-terpenes, aromatic, amines etc. The aroma-compound may also be called an odorant, aroma, or fragrance. The aroma-compound in the fragrance reservoirs may be stored as a solid (e.g. a powder), gel and/or liquid (e.g. in form of stock solutions).

Additionally to the at least one aroma-compound, the fragrance reservoir may comprise at least one non-olfactory ingredient, in particular at least one solvent (e.g. alcohol like ethanol or such, glycol and/or water) and/or at least one fixative. Thus, the fragrance reservoir may comprise at least one pure aroma-compound or at least one diluted aroma-compound. In particular, the fragrance reservoir may contain a stock solution of the at least one aroma-compound. The stock solution may refer to a concentrated solution that will be diluted by adding solvent to some lower concentration for actual use in the fragrance blend.

In particular, depending on its dilution, the fragrance blend may be a perfume (from 20 vol.-% to 40 vol.-% of stock solution diluted in ethanol and/or water as solvents), perfume water (from 10 vol.-% to 20 vol.-% of stock solution diluted in ethanol and water), toilet water (from 5 vol.-% to 10 vol.-% of stock solution diluted in ethanol and water), cologne (from 3 vol.-% to 5 vol.-% of stock solution diluted in ethanol and water), cologne water (from 1 vol.-% to 3 vol.-% of stock solution diluted in ethanol and water), or fragrance mist and air freshener (with less than 1 vol.-% of stock solution diluted in ethanol and water), etc. In particular, the fragrance blend is not intended to be used for food or other alimentary purposes.

A scent family may be a group of olfactory notes of a fragrance wheel that give a similar or common olfactory impression, e.g. a “fresh”, “floral”, “oriental”, or “woody”. The fragrance wheel may be a fragrance wheel according to Michael Edwards. For example, the floral family may be divided into the notes “floral” and “soft floral”. The fresh family may be divided into the notes “aromatic”, “citrus fruity”, “green”, “fruity”, and “water”. The oriental family may be divided into the notes “floral oriental”, “soft oriental”, “oriental”, and “woody oriental”. The woody family may be divided into the notes “woods”, “mossy woods”, and “dry woods”.

It is an embodiment that the scent families include a “fruity” scent family that comprises the notes “citrus fruity” and “fruity”. The fresh scent family then only comprises the aromatic, green, and water notes. It is thus an embodiment that the scent families comprise at least two scent families of a group comprising a floral, fresh, oriental, woody, and fruity scent family.

The fragrance dispensing apparatus may query to select at least one further fragrance reservoir that contains at least one aroma-compound that belongs to a note that is necessary to complete all levels of a scent pyramid. In one embodiment, the scent pyramid comprises three levels. The three levels may comprise a “top” level comprising “top” notes that appear first upon application. The top notes may e.g. comprise the fruity note and the aromatic note. The three levels may further comprise a “middle” level comprising “middle” notes that are the “heart” of a fragrance blend and that appear when the top notes fade. The middle notes may e.g. comprise the floral note, the green note, the fruity note, and a spicy note. The three levels may further comprise a “base” level comprising “base” notes that appear last but stay up to 24 hours. The top notes may e.g. comprise the woody notes, and a balsam note.

It is an embodiment that the method comprises querying to select a fragrance reservoir containing several aroma-compounds belonging to a common first scent family and a fragrance reservoir containing several aroma-compounds belonging to a common second scent family. Thus, the fragrance blend comprises at least two aroma-compounds each from at least two scent families. This gives the advantage that a particularly complex fragrance blend can be easily selected by a user.

It is an embodiment that at least one fragrance reservoir contains several aroma-compounds belonging to a common scent family and to at least two levels of the scent pyramid. If thus one of the scent families comprises two aroma-compounds belonging to two different levels of the scent pyramid and one other scent family comprises an aroma-compound belonging to the other, “missing” level of the scent pyramid, a fragrance blend with a well-rounded smell can be mixed by using only two fragrance reservoirs. Of course, a user may select to add further aroma-compounds from additional fragrance reservoirs.

It is an embodiment that a least one fragrance reservoir contains several aroma-compounds belonging to all levels of notes. This may further reduce the number of fragrance reservoirs and add to a well-rounded smell of the fragrance blend.

The object is further achieved by a set of fragrance reservoirs wherein at least two fragrance reservoirs contain pre-blend mixtures of aroma-compounds and wherein the aroma-compounds of a first fragrance reservoir all belong to a first scent family and the aroma-compounds of a second fragrance reservoir all belong to a second scent family and the first family and the second family are different. A pre-blend mixture may be understood to describe a mixture of aroma-compounds that lacks at least one aroma-compound to form the final fragrance blend.

The object is also achieved by a fragrance dispensing apparatus for dispensing a user-selected fragrance blend which apparatus is adapted to perform the method described above. This fragrance dispensing apparatus achieves the same advantages as the method and can be embodied in analogy to the method.

The fragrance dispensing apparatus may be a household appliance.

The object is particularly achieved by a fragrance dispensing apparatus, comprising several fragrance reservoirs containing aroma-compounds, an identification means for identifying the aroma-compounds, a user interface for selecting at least two fragrance reservoirs containing aroma-compounds to be blended into the fragrance blend, at least one extraction means for extracting the aroma-compounds from the selected fragrance reservoirs, and a fragrance dispensing means or “fragrance dispenser” for dispensing the fragrance blend comprising the extracted aroma-compounds, wherein: at least one first fragrance reservoir contains several aroma-compounds belonging to a common first scent family, at least one second fragrance reservoir contains at least one aroma-compound belonging to a common second scent family, and the user interface is adapted to query a user to select fragrance reservoirs such that the fragrance blend comprises aroma-compounds belonging to all groups of a scent pyramid.

The fragrance dispensing apparatus may be adapted to store information regarding a connection between the identified aroma-compounds and their respective fragrance reservoirs. Identifying the aroma-compounds may include reading information regarding the aroma-compounds from the fragrance reservoirs and/or identifying the fragrance reservoirs.

It is an embodiment that the fragrance dispensing apparatus comprises at least one solvent reservoir for at least one solvent. The fragrance dispensing apparatus further comprises at least one extraction means for extracting at least one solvent from at least one solvent reservoir. In particular, the user interface may be adapted query to select a type of the fragrance blend (e.g. perfume, cologne etc.). The amount of solvent necessary to achieve the selected type to the fragrance blend may then automatically be added to the fragrance blend. The provision of the at least one solvent reservoir gives the advantage that a type of fragrance blend can be selected instantly (e.g., perfume, perfume water, toilet water, cologne, cologne water, fragrance mist etc.). Additionally, a volume of the fragrance reservoirs can be kept small that way, enabling a compact design and/or a larger number of fragrance reservoirs.

It is an embodiment that the reservoirs (i.e. fragrance reservoirs and/or solvent reservoir(s), if available) are disposably insertable into the fragrance dispensing apparatus. A reservoir may thus be a disposable or exchangeable reservoir. In this case, the reservoir may also be called a cartridge. The cartridge may comprise exactly one reservoir and may then also be called a capsule. Alternatively, several fragrance reservoirs containing different aroma-compounds are positioned in a common disposable cartridge/the cartridge may comprise several reservoirs (e.g. several capsules) containing different aroma-compounds.

The fragrance dispensing apparatus may comprise at least one seat for several reservoirs, e.g. at least one seat for one or more (multi-reservoir) cartridges and/or several seats for capsules (single-reservoir cartridges). The seat may also be called a compartment, cavity or support.

At least one reservoir may alternatively be fixedly positioned in the fragrance dispensing apparatus. In this case, the reservoir may be called a tank. Such a tank may be refillable by a user. A solvent tank may contain one solvent (e.g. water, alcohol, glycerine) or a solvent mixture (e.g. a mixture of water and alcohol). The reservoirs, and in particular a tank, may be equipped with a level sensor. The fragrance dispensing apparatus may comprise at least one seat for at least one removable fragrance reservoir and at least one tank for at least one solvent. In particular, the fragrance dispensing apparatus may comprise at least one seat for a fragrance cartridge and at least one solvent tank.

In one embodiment at least one seat or reservoir is connectable or connected to a respective extraction means via a respective coupling means (also called a connector). The coupling means is adapted to prevent unwanted spill or leakage from the respective reservoir and to connect the reservoir to the extraction means. The coupling means may be opened to generally allowing discharging the reservoir's content.

In one variant, the coupling means comprises a valve. The valve may e.g. be a one-way valve. For example, the valve may be a pressure operated valve, a pinch valve, a needle valve etc.

In another variant, the reservoir's outlet is sealed with a rubber cap that works as dropper or dosage means.

In yet another variant, the hollow needle can be inserted into the reservoir through the rubber cap to extract content from the reservoir. The rubber cap makes a tight connection with the needle, and once the needle is pulled out of the reservoir, the rubber cap recovers its shape thanks to its elastic properties thus avoiding spillage of the remaining content. The needle may be a part of the extraction means while the rubber seal may be regarded as the coupling means.

The extraction means (or “extractor”) may comprise at least one dosing means to precisely control the amount (e.g. weight, flow volume etc.) of the aroma-compound(s) and—if available—solvent(s) dispensed or extracted from the reservoirs. The dosing means may comprise or be a precisely controllable dosage valve.

The extraction means may further comprise a pump for pumping the aroma-compound(s) and possibly solvent(s) from the reservoirs. This supports a constant and precise flow of the reservoir's content. For example, the pump may be a peristaltic pump.

Additionally, the extraction means may comprise a flow meter for measuring the amount of aroma-compound(s) and possibly solvent(s) dispensed or extracted. This allows a particularly precise measurement and extraction of the reservoir's content.

Regarding a flow direction of the reservoir's contents, the extraction means is positioned between at least one reservoir and the fragrance dispenser.

It is an embodiment that each reservoir is connected to a respective extraction means (e.g. via a respective coupling means). This enables a particularly precise extraction or dosing and avoids an unwanted mixture of aroma-compounds.

It is another embodiment that at least one extraction means is simultaneously/parallelly connectable or connected only to at least one fragrance reservoir (e.g. via a respective coupling means) and at least one other extraction means is simultaneously connectable or connected only to at least one solvent reservoir (e.g. via a respective coupling means). This allows a precise and fast dosing and reduced costs. In particular, an extraction means connectable to a solvent reservoir may have a greater flow-rate than an extraction means connectable to a fragrance reservoir which allows fast dispensing of the solvent. This makes use of the fact that a solvent quantity needed for the fragrance blend is typically much higher than the quantity needed of the fragrances. In one variant, exactly one extraction means is connected or connectable to the fragrance reservoir(s) and exactly one other extraction means is connected or connectable to the solvent reservoir(s). Both extraction means may comprise a respective pump and flow meter.

It is yet another embodiment that at least one extraction means is simultaneously connectable or connected to at least one fragrance reservoir and additionally connectable or connected to at least one solvent reservoir. This allows a particularly cost effective extraction. In particular, the at least one extraction means comprises exactly one extraction means that is connectable or connected to all reservoirs.

The connection between the extraction means and a reservoir, its seat, or its valve, respectively, may comprise using a tube, pipe, duct or other conduit.

It is even another embodiment that the at least one extraction means comprises an extractions means for consecutively being connectable or connected to several reservoirs. For example, such an extraction means may comprise a needle. By moving the needle and/or the reservoirs, the needle may be consecutively inserted into selected reservoirs to extract the respective aroma-compound(s) and/or possibly solvent(s). The movement may e.g. be performed by a step motor. In particular, this embodiment comprises exactly one extraction means which allows an especially cost-effective setup. Also, the coupling means may not be needed/avoided in this embodiment. For example, the reservoirs may comprise rubber seals hermetically sealing an opening to allow a tight fit of the needle and a reliable closure after the needle has been removed.

It is yet another embodiment that the fragrance dispensing apparatus comprises a blending chamber for blending the extracted aroma-compounds and possibly the at least one solvent into a fragrance blend. If the blending chamber is present, the pump may be used for pumping the aroma-compound(s) and possibly solvent(s) from the reservoirs to the blending chamber. Regarding a flow direction of the reservoir's contents, the extraction means may be positioned between at least one reservoir and the blending chamber.

It is an embodiment thereof that the blending chamber is a “static” chamber that simply holds the extracted aroma-compound(s) and solvent(s). This is a particularly cost-effective embodiment. The aroma-compound(s) and/or solvent(s) may be kept for a predefined time to enhance the homogeneity of the fragrance blend.

It is another embodiment thereof that the blending chamber is a “continuous” chamber in that the aroma-compound(s) and/or solvent(s) are blended based on a loss of pressure when they are flowing together through the blending chamber.

It is yet another embodiment thereof that the blending chamber is a “bubbling” chamber which is based on creating bubbles at the bottom of the blending chamber that will circulate upwards through all the aroma-compound(s) and/or solvent(s) thus speeding up the blending process. The bubbles may be created by an air pump and e.g. membranes at the bottom of the blending chamber.

It is even another embodiment thereof that the blending chamber comprises a mixing blade. The mixing blade may be driven by a rotor. Such a blending chamber works with powder-liquid and liquid-liquid blends. In particular, the blending chamber may form a close-clearance mixer. There are several types of close-clearance mixers, most common ones use mixing blades in form of anchors or helical ribbons. Anchor mixers induce solid-body rotation but do not promote vertical mixing, while helical ribbons do promote vertical mixing. Other types of close-clearance mixers may use Rushton blades, hydrofoil blades, radial blades, axial blades etc.

It is an embodiment that the fragrance dispensing apparatus comprises an identification means for identifying the reservoirs, in particular cartridges, in particular fragrance cartridges, and the contained aroma-compounds, respectively. This facilitates replacing or newly inserting cartridges into the fragrance dispensing apparatus. To this effect, identification technologies like optical reading (e.g. barcode scanning), RFID querying (e.g., NFC) and so on can be used.

In one variant, the identification means (or “identificator”) comprises an optical code reader (e.g. light and/or IR scanner) to read a code (e.g. a barcode or a QR code) of the cartridges. For example, there is provided one code reader for each cartridge to identify the cartridge's content. The readers may be placed at a certain distance from each cartridge. This is a particularly affordable variant if the fragrance dispensing apparatus may only contain a small number of cartridges.

In another variant, the identification means comprises a wireless radio frequency reader, e.g. a RFID reader, in particular an NFC reader. This variant has the advantage that one reader may be sufficient to read respective RFID tag of multiple, in particular all, cartridges. The cartridges are consequently equipped with such RFID tags. This variant has the further advantage that a distance and orientation of the RFID reader with respect to the RFID tags may be more flexible.

The reader(s) may be placed at a fixed location/fixed locations within the fragrance dispensing apparatus. In an alternative, a reader may be moved or movable within the fragrance dispensing apparatus such that the reader may be placed in front of each code/tag. This is particularly advantageous when using an optical reader. Alternatively, the cartridges may be moved to the fixed reader. This identification means may comprise a rotor that rotates the seats of the cartridges or that rotates the reader for a direct reading, in particular optical reading.

The fragrance dispenser may also be called a fragrance dispensing means, a fragrance dispensing module or a fragrance dispensing component.

It is an embodiment that the fragrance dispenser is adapted to apply the aroma-compound(s) of at least one fragrance reservoir directly (i.e., circumventing the blending chamber, if present) to a test substrate and providing this test substrate. This allows a customer to smell the content of the fragrance reservoir before ordering the fragrance dispensing apparatus to process the final fragrance blend.

In one variant, the test substrate is a test strip, e.g. made of blotting paper. In this case, the fragrance dispensing apparatus may comprise a test strip dispenser where the test strip is provided with (e.g. sprayed with) the content of one or more fragrances reservoirs. The customer can take the strip to smell the sample.

In another—even simpler—variant, the customer takes one test strip from the test strip dispenser and rubs it against a surface that contains the sample.

The fragrance dispensing apparatus may be adapted to allow a customer to smell the aroma-compounds of the fragrance reservoir the customer wants to add to the fragrance blend. To this effect, the extraction means may be adapted to put a drop of the requested content onto the test strip. The customer can take the test strip and smell it.

Additionally or alternatively, the fragrance dispensing apparatus may be adapted to allow a customer to smell the content of multiple fragrance reservoirs. In this case, to achieve a good approximation of the desired fragrance blend, the extraction means may extract and dispense the aroma-compounds from the multiple fragrance reservoirs in the right proportions. The customer may shake the test strip a little bit to let the ingredients mix.

In yet another variant, the test substrate is housed in a tube (“test tube”). The test substrate may be ceramic wool or any other suitable absorbent material. The aroma-compound(s) associated with the selected fragrance reservoir(s) is or are dispensed into the test tube. The test tube may comprise a heater that heats the ceramic wool once the customer wants to smell the sample. The heater may be working until the customer wants to test another fragrance reservoir or until the aroma-compounds have totally evaporated. The test tube may be part of the fragrance dispensing apparatus and may not be removable.

The fragrance dispenser may e.g. dispense the test mix as a liquid or as a spray.

It is an embodiment that the fragrance dispenser is adapted to dispense the aroma-compound(s) of the selected fragrance reservoir(s) and possibly the solvent(s) into a withdrawable container, e.g. into a bottle (“fragrance bottle”). Fragrance bottles have the advantage to have a perceived high value. The fragrance dispenser may comprise a flow meter to precisely measure the amount of the fragrance blend let into the withdrawable container. Additionally, the fragrance dispenser may comprise a pump to pump the fragrance blend into the withdrawable container.

In particular for the case that the fragrance dispensing apparatus comprises a blending chamber, the fragrance dispenser may be adapted to dispense a ready fragrance blend from the blending chamber into the withdrawable container. In this embodiment, the fragrance dispenser may comprise a valve, in particular a dosage valve, to let the ready fragrance blend out of the blending chamber. The fragrance dispenser may comprise a flow meter to precisely measure the amount of the fragrance blend let out of the blending chamber and into the withdrawable container. Additionally, the fragrance dispenser may comprise a pump to pump the fragrance blend from the blending chamber into the withdrawable container. Alternatively, and particular for the case that the fragrance dispensing apparatus does not comprise a blending chamber, the fragrance dispenser is adapted to dispense the aroma-compound(s) from the selected fragrance reservoir(s) and the solvent(s) directly into a withdrawable container, e.g. into a bottle. In one variant, the fragrance dispenser is adapted to dispense the liquid aroma-compounds into a bottle that changes its colour when being filled. The bottle may e.g. be covered with a PVD (Physical Vapour Deposition) coating that allows light emitted from/trough the bottle to change its colour while it is being filled with the fragrance blend. The bottle may be of customized design. In another variant, the fragrance bottle is designed such that it uses or exhibits the Coanda effect to generate an attractive visual effect for the customer once the solution is being dispensed. To this effect, the bottle comprises a translucent (e.g. transparent) inner curved surface that is wetted by the liquid fragrance blend when it is dispensed into the bottle. Additionally, the fragrance dispensing apparatus—and in particular its fragrance dispenser—comprises a light projector to project light of different colours through this curved surface. This generates different light effects when the light passes through the moving liquid.

It is an additional or alternative embodiment that the fragrance dispenser comprises a fragrance spraying device to directly spray the ready fragrance blend from the fragrance dispensing apparatus. The fragrance spraying device may comprise a spray nozzle. The spray nozzle may be activated by a customer, e.g. being mechanically activated by pressing it. Therefore, the fragrance dispenser may be adapted to dispense the selected aroma-compound(s) and the solvent(s) through the spray nozzle. This can be achieved directly or via the blending chamber.

It is an embodiment that the fragrance dispensing apparatus is a self-cleaning device. This enables to conveniently remove aroma-compound(s) and/or solvent(s) that have been left over from a dispensing process. Such leftover aroma-compound(s) and/or solvent(s) may rest in pipes, pumps, the blending chamber etc., or even external components. The fragrance dispensing apparatus may start a self-cleaning process, e.g. automatically and/or by user command. For example, the self-cleaning process may dispense a certain amount of solvent (e.g. ethanol) in order to dissolve and remove leftover traces of aroma-compounds. In one variant, at least one solvent used for creating the fragrance blend may be used. This is typically sufficient since most aroma-compounds are soluble. In an alternative or additional variant, dedicated cleaning chemicals may be used, e.g. contained in a specific cleaning reservoir.

To avoid or slow down a degradation of the aroma-compounds, fragrance reservoirs with a reduced amount of oxygen may be used. Rather, the fragrance reservoirs may comprise a protective atmosphere, e.g. made up of nitrogen and/or carbon dioxide. Also, the fragrance bottles given out from the fragrance dispensing apparatus may comprise the fragrance blend in such a protective atmosphere.

It is an embodiment that the fragrance dispensing apparatus comprises a control device (e.g. a microcontroller, control module, control board, etc.) to operate the fragrance dispensing apparatus according to selections made by a user at the user interface. The control device may e.g. operate one or more from the group containing the extraction means, the blending chamber, the fragrance dispenser etc.

The above described features and advantages of the invention as well as their kind of implementation will now be schematically described in more detail by at least one embodiment in the context of one or more FIGURES of the attached drawing.

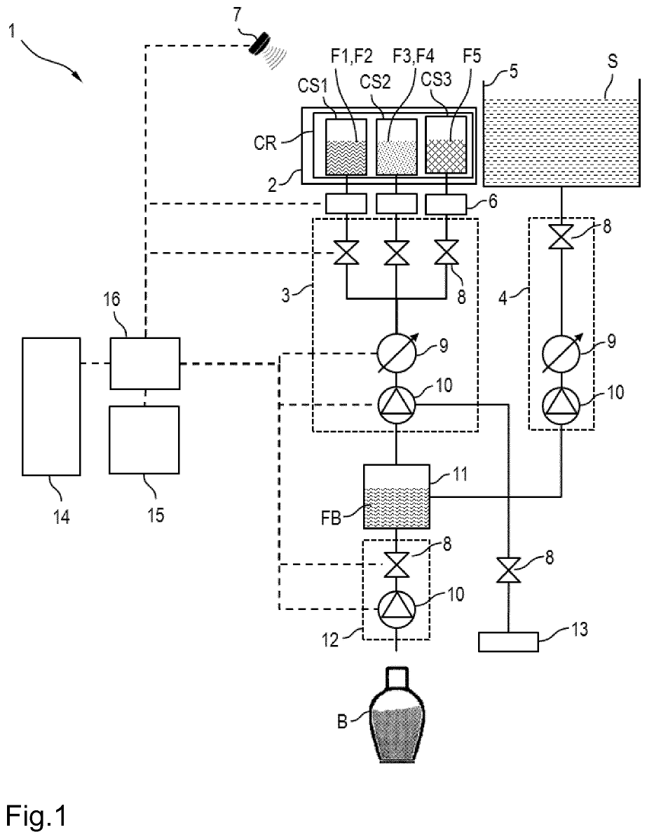

FIG. 1 shows a schematic drawing of a fragrance dispensing apparatus 1. The fragrance dispensing apparatus 1 comprises a seat 2 for a cartridge CR. The cartridge CR exemplarily comprises three fragrance reservoirs in form of capsules CS1, CS2, CS3. A first capsule CS1 contains a stock solution comprising at least two aroma-compounds F1, F2 of a first scent family (and no aroma-compound from another scent family). A second capsule CS2 contains a stock solution comprising at least two aroma-compounds F3, F4 of a second scent family (and no aroma-compound from another scent family). A third capsule CS3 contains a stock solution comprising at least one aroma-compound F5 of a third scent family (and no aroma-compound from another scent family). For example, the aroma-compounds F1 and F2 may belong to citrus fruity notes of the fruity scent family and are top notes with respect to the scent pyramid. The aroma-compounds F3 and F4 may belong to floral and/or soft floral notes of the floral scent family and are middle notes with respect to the scent pyramid. The aroma-compound F5 may belong to a woody or an oriental note and belongs to a base note with respect to the scent pyramid. Alternatively, the aroma-compounds F1 and F2 may belong to a citrus fruity note and a fruity note, respectively, of the fruity scent family and are thus a top note and a middle note, respectively. Also alternatively, the aroma-compounds F3 and F4 may belong to an aromatic note and a green note, respectively, of the fresh scent family and are thus a top note and a middle note, respectively.

The fragrance dispensing apparatus 1 may comprise further capsules (not shown) comprising aroma-compounds belonging to other scent families, e.g. the fresh scent family, and/or belonging to the same scent families, e.g. other aroma-compounds belonging to the fruity, floral, oriental and/or woody scent families. The further capsules may be part of the same cartridge CR or of an additional cartridge (not shown).

The fragrance dispensing apparatus 1 further comprises one extraction means 3 for selectively extracting contents from the respective capsules CS1 to CS3 and one extraction means 4 for extracting a solvent S (e.g. an ethanol/water mix) from a solvent tank 5. While the cartridge CR is removable by a user, the solvent tank 5 is fixedly positioned in the fragrance dispensing apparatus 1. The capsules CS1 to CS3 and/or the solvent tank 5 may be equipped with a level sensor (not shown).

The outlets of the capsules CS1 to CS3 are each connected to the extraction means 3 via respective coupling means 6 or connectors, e.g. in form of one-way valves. The extraction means 3 can selectively extract the contents from the respective capsules CS1 to CS3.

To automatically identify the capsules CS1 to CS3 and their respective aroma-compounds F1 to F5, the fragrance dispensing apparatus 1 comprises an identification means in form of an optical or radio frequency reader 7 that reads a respective code or tag of the cartridge CR. The queried information may include the kind of aroma-compound(s) F1 to F5, amount of the ingredients, dilution, etc. contained in the capsules CS1 to CS3. Alternatively or additionally, a code or tag may be associated with each of the capsules CS1 to CS3.

The extraction means 3 comprises dosage valves 8 that are connected to a respective coupling means 6. The dosage valves 8 enable extracting a very precise amount of aroma-compounds F1 to F5. The dosage valves 8 are connected to a flow meter 9 which, in turn, is connected to a pump 10. The pump 10 is connected to a blending chamber 11. The blending chamber 11 is also connected to the solvent tank 5 via the extraction means 4. While the extraction means 4 may have a generally similar setup (e.g. comprising a dosage valve 8, a flow meter 9, and a pump 10) as the extraction means 3, it may have a larger flow-rate than the extraction means 3. Also, there may not be a coupling means 6 between the solvent tank 5 and the extraction means 4.

The blending chamber 11 is used to blend the aroma-compounds F1 to F5 extracted from the capsules CS1 to CS3 and possibly the solvent S extracted from the solvent tank 5 to create a fragrance blend FB. The blending chamber 11 may be a static chamber, a continuous chamber, a bubbling chamber, a mixing chamber (e.g. a close-clearance mixer), etc.

Further, the blending chamber 11 is connected to a fragrance dispenser 12 for dispensing the blended fragrance mixture FB from the blending chamber 11 into a fragrance bottle B. The fragrance dispenser 12 may comprise a dosage valve 8, a pump 10, and/or a flow meter (not shown). Alternatively or additionally, the fragrance dispenser 12 may comprise a fragrance spraying device (not shown), e.g. a spray nozzle.

An output side of the extraction means 3 is also connected to a test strip dispenser 13 via an optional dosage valve 8 for dispensing the content of at least one capsule CS1 to CS3 onto a substrate in form of a test strip (not shown) and providing the test strip. A pump and/or a flow meter (not shown) may also be present between the extraction means 3 and the test strip dispenser 13. Thus, corresponding aroma-compounds F1 to F5 are directly applied to the test strip. The fragrance dispensing apparatus 1 is adapted to selectively extract the content of the capsules CS1 to CS3 towards the blending chamber 11 or the test strip dispenser 13. Alternatively or additionally to the test strip dispenser 13, a spray nozzle may be used. Alternatively to the test strip dispenser 13, the fragrance dispensing apparatus 1 may comprise a heatable open test tube (not shown) that contains a substrate for the aroma-compounds F1 to F5 dispensed from the extraction means 3. To this effect, a 3/2-way valve (not shown) or such may be used.

The fragrance dispensing apparatus 1 further comprises a user interface 14 for selecting capsules CS1 to CS3 until aroma-compounds F1 to F5 belonging to all levels of a scent pyramid have been selected.

Additionally, the fragrance dispensing apparatus 1 comprises a communication module 15, e.g. for communicating a need to replace the cartridge CR or to fill up the solvent tank 5.

Moreover, the fragrance dispensing apparatus 1 comprises a control device 16 (e.g. a control module comprising a microcontroller, an ASIC, a FPGA etc.) to operate or use the fragrance dispensing apparatus 1, e.g. some or all of its electrically controllable components 3 to 4, 5 (its level sensor), 6 to 14. For example, by activating the reader 7, the control device 16 identifies the available aroma-compounds F1 to F5 and may calculate their remaining amount. Also, the control device 16 has information about the available solvent S and its amount. The control device 16 may thus present available selections/choices in the user interface 14.

In one possible embodiment, if a user wants to receive a fragrance blend FB, the user is informed by the user interface 14 about the available scent families, notes, their levels and/or the respective aroma-compounds F1 to F5. The user may then select a scent family, note or aroma-compound F1 to F5. If the user has selected a certain scent family, the user interface 14 may show the corresponding capsules CS1 to CS3 and their aroma-compounds F1 to F5. If there is more than one capsule CS1 to CS3 available for the selected scent family, the user interface 14 may ask the user to select one of these capsules CS1 to CS3. After having selected one scent family, one corresponding capsule CS1 to CS3 and one corresponding aroma-compound F1 to F5, respectively, the fragrance dispensing apparatus 1 automatically determines for which scent levels of a scent pyramid no aroma-compounds F1 to F5 have been selected yet. The user is then shown—by the user interface 14—capsules CS1 to CS3 (and their aroma-compounds F1 to F5, notes and/or levels) that comprise such missing aroma-compounds F1 to F5. Then, the user may be asked to select one of these capsules CS1 to CS3. If there is still a scent level missing, this step may be repeated until the selected capsules CS1 to CS3 comprise aroma-compounds F1 to F5 of all levels of the scent pyramid. The user interface 14 may ask the user to add the content of further capsules CS1 to CS3, if so desired.

Additionally, the user interface 14 may query the user for the desired amount (e.g. 25 ml) and/or type (e.g. perfume) of the fragrance blend FB.

The control device 16 opens the respective coupling means 6 and operates the respective dosing valve(s) 8 of the extraction means 3 to extract the selected amount or quantity from the respective capsules CS1 to CS3, e.g. one after another. In one embodiment, the relative amount extracted from the respective capsules CS1 to CS3 is automatically determined by the fragrance dispensing apparatus 1, e.g. on the basis of experimentally determined ratios. Such ratios may be stored in a lookup table. In one variant, the user interface 14 may allow the user to alter the automatically determined relative amount. This gives the advantage that a user may easily emphasize certain scent families, notes and/or aroma-compounds F1 to F5. It is also a variant that a user may freely input a desired (absolute or relative) amount of the contents of the different capsules CS1 to CS3 and thus their aroma-compounds F1 to F5. The pump 10 of the extraction means 3 then pumps the corresponding contents of the different capsules CS1 to CS3 into the blending chamber 11.

Similarly, the control device 16 commands the extraction means 4 to extract a certain quantity of the solvent S into the blending chamber 11. The quantity of the solvent S is determined by the control device 16 on basis of the selected type of fragrance blend FB. The quantities of the capsules' CS1 to CS3 content and the solvent S are measurable by the flow meter(s) 9. The control device 16 may also operate the blending chamber 11, if possible. Moreover, the control device 16 operates the fragrance dispenser 12 to dispense the ready fragrance blend FB, e.g. into a bottle B or onto a test strip etc.

The fragrance dispensing apparatus 1 may be a self-cleaning device.

Of course, the invention is not restricted to the described embodiments.

For example, the user interface 14 may generally show descriptions of capsules CS1 to CS3 (e.g. their scent families, notes, aroma-compounds F1 to F5, information regarding the aroma-compounds F1 to F5 etc.). The user interface 14 may also show general information regarding scent families, scent pyramids, aroma-compounds F1 to F5 and so on, e.g. in form of an interactive database or lexicon.

### LIST OF REFERENCE NUMERALS

1 fragrance dispensing apparatus
2 seat
3 extraction means
4 extraction means
5 solvent tank
6 coupling means
7 reader
8 dosage valve
9 flow meter
10 pump
11 blending chamber
12 fragrance dispenser
13 test strip dispenser
14 user interface
15 communication module
16 control device
B bottle
CR cartridge
CS1-CS3 capsules
FB fragrance blend
F1-F5 aroma-compound
S solvent

1. A method for providing a user-selected fragrance blend by a fragrance dispensing apparatus, the method comprising:
querying to select, via a user interface, a fragrance reservoir containing several aroma-compounds belonging to a common scent family, and
querying to select, via the user interface, at least one other fragrance reservoir containing at least one aroma-compound belonging to another scent family, until aroma-compounds belonging to all levels of a scent pyramid have been selected,
extracting the aroma-compounds from the selected fragrance reservoirs, and
dispensing the extracted aroma-compounds as a fragrance blend.

2. The method according to claim 1 comprising querying to select a fragrance reservoir containing several aroma-compounds belonging to a common first scent family and a fragrance reservoir containing several aroma-compounds belonging to a common second scent family.

3. The method according to claim 1 wherein at least one fragrance reservoir contains several aroma-compounds belonging to a common scent family and to at least two levels of the scent pyramid.

4. The method according to claim 1 wherein the scent families comprise at least two scent families of a group comprising a fresh scent family, a fruity scent family, a floral scent family, an oriental scent family, and a woody scent family.

5. The method according to claim 1 wherein at least one fragrance reservoir contains several aroma-compounds belonging to all levels of notes.

6. The method according to claim 1 further comprising: querying to select, via the user interface, an amount of the aroma-compounds from different fragrance reservoirs, and adding the aroma-compounds to the fragrance blend in accordance to the selected amount.

7. The method according to claim 1 further comprising: querying to select, via the user interface, a type of the fragrance blend, and adding an amount of solvent to the fragrance blend in accordance to the selected type.

8. A fragrance dispensing apparatus for dispensing a user-selected fragrance blend, comprising:
several fragrance reservoirs containing aroma-compounds,
an identification means for identifying the aroma-compounds stored in the fragrance reservoirs,
a user interface for selecting at least two fragrance reservoirs containing aroma-compounds to be blended into the fragrance blend,
at least one extraction means for extracting the aroma-compounds from the selected fragrance reservoirs, and
a fragrance dispenser for dispensing the fragrance blend comprising the extracted aroma-compounds,
wherein
at least one first fragrance reservoir contains several aroma-compounds belonging to a common first scent family,
at least one second fragrance reservoir contains at least one aroma-compound belonging to a common second scent family, and
the user interface is adapted to query a user to select fragrance reservoirs such that the fragrance blend comprises aroma-compounds belonging to all groups of a scent pyramid.

9. The fragrance dispensing apparatus according to claim 8, wherein the fragrance reservoirs are disposably insertable into the fragrance dispensing apparatus.

10. The fragrance dispensing apparatus according to claim 8, wherein several fragrance reservoirs are positioned in a common disposable cartridge.
